(19) United States
(12) Patent Application Publication
(10) Pub. No.: US 2005/0045185 A1
(43) Pub. Date: Mar. 3, 2005

(54) Emission bag

(76) Inventor: Gary S. Takowsky, Beverly Hills, CA (US)

Correspondence Address: TOPE-MCKAY & ASSOCIATES, 23852 PACIFIC COAST HIGHWAY #311, MALIBU, CA, 90265, US

(21) Appl. No.: 10/651,530

(22) Filed: Aug. 29, 2003

(51) Int. Cl. A61F 9/00

(52) U.S. Cl. 128/858

(57) ABSTRACT

The present invention relates to a barrier, and more particularly, to an emission bag where a user coughs or sneezes into the emission bag, with the emission bag acting as a barrier such that pathogens transmitted from a nose or mouth will be confined to an interior of the emission bag. The emission bag has small perforations allowing it to breathe, so that when a user coughs or sneezes into the emission bag, it doesn't immediately fill full of air and cause a blow-back of the pathogens onto a user's face. Additionally, the small perforations are medicated with an anti-septic, such that emissions are sterilized upon passing through the small perforations.

### FIELD OF INVENTION

The present invention relates to a barrier, and more particularly, to an emission bag where a user coughs or sneezes into the emission bag, with the emission bag acting as a barrier such that pathogens ejected from a nose or mouth will be confined to an interior of the emission bag. Additionally, the emission bag has small perforations that are medicated with an anti-septic, such that emissions are sterilized upon passing through the small perforations.

### BACKGROUND OF INVENTION

Pathogens are often transmitted through fluid droplets, such as mucous, saliva, and nasal secretions. Projected through a cough or sneeze, the droplets act as carriers, transferring the pathogen from one individual to another. A single cough may produce anywhere from a few hundred to several thousand droplets, each potentially contaminated with pathogens. Contrasted with a sneeze, a sneeze may produce anywhere from a few hundred to a few million droplets. While some of the droplets produced by a sneeze may be large enough to immediately fall to the floor, others evaporate, forming residues of “droplet nuclei” which may remain airborne for hours or even days.

Preventing transmission of pathogens is often accomplished through use of barriers. The barriers are used to prevent the droplets from transferring to one's hand or from becoming airborne. The barriers take form as rubber gloves, handkerchiefs, tissues, and mitts. While effective in a sterile setting such as a hospital, rubber gloves are impractical for daily use. If an individual were to sneeze or cough, she would unlikely place a rubber glove over her hands before sneezing into them. While more practical than rubber gloves, handkerchiefs and tissues do little to prevent contamination of the hands since the microorganisms progress through the barrier from one site to another.

In an attempt to prevent contamination of the hands, a few inventors have created mitts with impervious layers. As disclosed in U.S. Pat. Nos. 5,196,244 and 5,864,883, to Beck and Reo respectively, a mitt-like bag couples an impervious inner layer with an absorbent external layer. As disclosed in Beck and Reo, a user inserts her hand into the mitt-like bag and proceeds to blow her nose, sneeze, or cough onto the absorbent external layer of the mitt-like bag. The user then turns the mitt-like bag inside-out, sealing any pathogens on a now interior portion.

Although the mitt-like bags are successful in preventing contamination of the hands, they are not entirely successful in preventing pathogens from becoming airborne. When sneezing directly onto the absorbent external layer of the mitt-like bag, it is likely that pathogens will escape. Additionally, because of the impervious inner layer, if a user were to sneeze directly into an internal portion of the mitt-like bag, air would quickly fill the mitt-like bag and cause a blow-back of pathogens onto the user's face.

Therefore, there exists a need to present a bag where a user may sneeze or cough directly into an internal portion of a sealable bag, the bag being such that it allows air to pass through the bag, while keeping droplets and pathogens in the internal portion. Additionally, because sneezes and coughs often come unexpectantly, it is desirable that the bag be small and portable, allowing a user to carry one bag or a package of several bags. In this regard, the present invention substantially fulfills this need.

### SUMMARY OF INVENTION

The present invention comprises an emission bag for covering a nose and mouth in such a manner that pathogens transmitted from the nose and mouth will be confined to an interior of the emission bag. The emission bag comprises a piece of material with an opening; and a bag attached with the piece of material and opening, such that an entrance to an interior of the emission bag is through the opening.

In another aspect, a top film is attached with the emission bag, such that manipulating the top film exposes the opening. Additionally, a bottom film is attached with a bottom portion of the piece of material, whereby manipulating the bottom film exposes the bag. In another aspect, the bottom film may also be attached with an exterior surface of the bag, such that pulling on the bottom film pulls open the bag and extends the bag away from the opening.

In another aspect, the piece of material is constructed from a material selected from a group consisting of plastic, metal and paperboard. Additionally, the piece of material may have perforations, allowing it to fold along the perforations and thereby create two opposing sides.

In yet another aspect, the piece of material has an enclosure apparatus, such that when the piece of material is folded along the perforations, the enclosure apparatus on the two opposing sides come into contact with each other and thereby seal the emission bag. The enclosure apparatus is selected from a group consisting of adhesive tabs, tape, Velcro, glue, and twist-ties.

Additionally, the bag is constructed of a material selected from a group consisting of plastic, paper and cloth.

In another aspect, the bag has small perforations allowing the bag to breathe. The small perforations are medicated with an anti-septic on the interior of the bag, such that emissions are sterilized upon passing through the small perforations.

Finally, the bag has an entrance and a base and the entrance has an area and the base has an area, where the area of the entrance is smaller than the area of the base.

### BRIEF DESCRIPTION OF THE DRAWINGS

The nature of the emission bag described herein will be readily apparent in the following drawings, in which:

FIG. 1 is a top perspective view of the present invention, showing a piece of material with an opening;

FIG. 2A is a cross-sectional view of the present invention, taken from line II-II of FIG. 1, showing the piece of material with a top film and a bottom film attached thereto;

FIG. 2B is a cross-sectional view of the present invention, taken from line II-II of FIG. 1, showing an emission bag attached with the piece of material; and

FIG. 3 is a cross-sectional view of the present invention, taken from line II-II of FIG. 1, showing the piece of material being folded, with two opposing sides coming into contact with each other and thereby sealing the emission bag.

### DETAILED DESCRIPTION

The present invention relates to a barrier, and more particularly, to an emission bag where a user coughs or sneezes into the emission bag, with the emission bag acting as a barrier such that pathogens transmitted from a nose or mouth will be confined to an interior of the emission bag. Additionally, the emission bag has small perforations that are medicated with an anti-septic, such that emissions are sterilized upon passing through the small perforations. An inherent advantage of this invention is that it is small and portable, allowing a user to carry a single emission bag or a package containing several emission bags.

The following description, taken in conjunction with the referenced drawings, is presented to enable one of ordinary skill in the art to make and use the invention. Various modifications will be readily apparent to those skilled in the art, and the general principles defined herein may be applied to a wide range of aspects. Thus, the present invention is not intended to be limited to the aspects presented, but is to be accorded the widest scope consistent with the principles and novel features disclosed herein. Furthermore it should be noted that unless explicitly stated otherwise, the figures included herein are illustrated diagrammatically and without any specific scale, as they are provided as qualitative illustrations of the concept of the present invention.

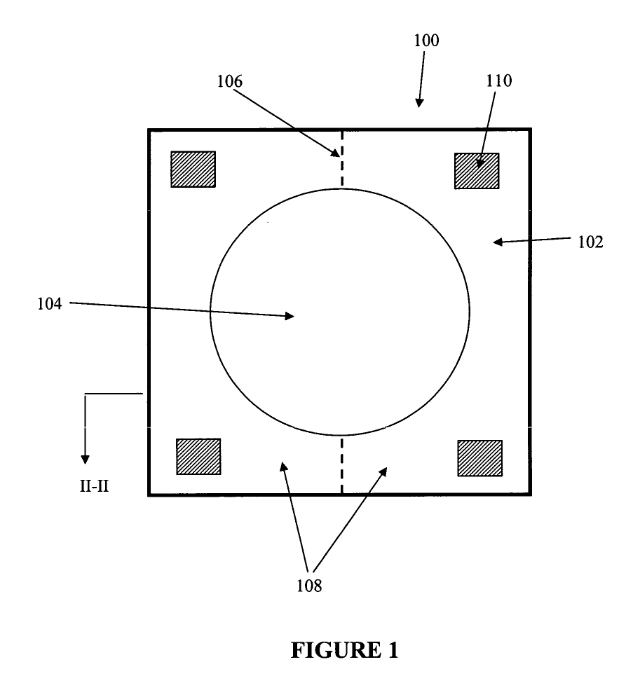

Referring to the figures, FIG. 1 illustrates an aspect of the emission bag 100 as piece of material 102 with an opening 104. The piece of material 102 may be constructed of any suitably semi-rigid material, non-limiting examples of which include plastic, metal and paperboard. For example, the piece of material 102 may be a planar board constructed of paperboard, with the opening as a hole in the planar board. Additionally, the piece of material 102 may be comprised of a single layer or multiple layers. The piece of material 102 has perforations 106 running from one side of the piece of material 102 to the other, allowing it to fold along the perforations 106 and thereby creating two opposing sides 108. An enclosure apparatus 110 is attached with the piece of material 102, such that when the piece of material 102 is folded along the perforations 106, the enclosure apparatus 110 on the two opposing sides 108 come into contact with each other and thereby seal the emission bag 100. The enclosure apparatus 110 may be any suitable apparatus for affixing two mediums together, non-limiting examples of which include adhesive tabs, tape, Velcro, glue, and twist-ties.

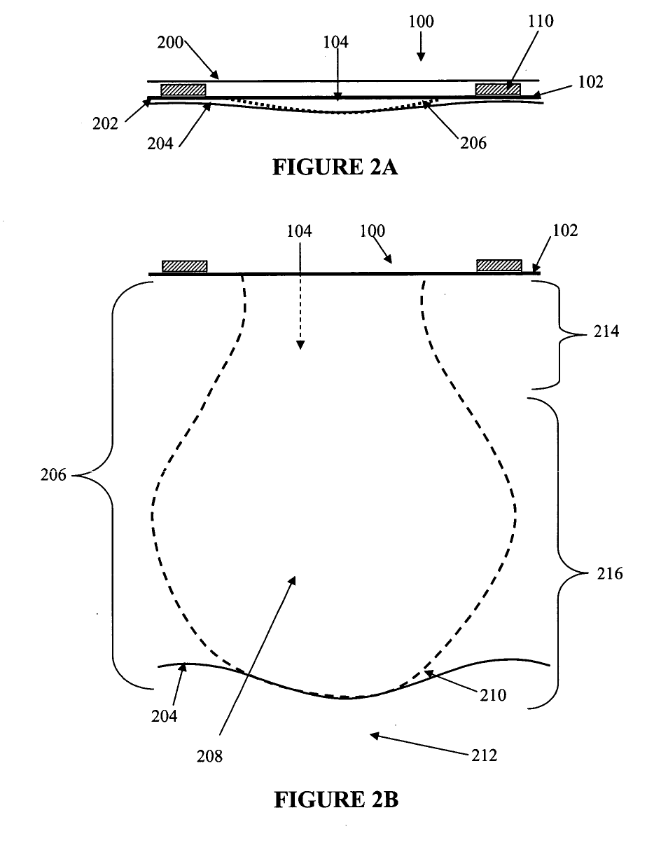

As illustrated in FIG. 2A, a top film 200 is attached with the emission bag 100, such that the top film 200 needs to be peeled off or otherwise manipulated in order to expose the opening 104. The top film 200 may be attached with the emission bag 100 through any suitable means for affixing two mediums together, non-liming examples of which include staples, tape, adhesive and being in-sewn. Additionally, the top film 200 may be attached with the enclosure apparatus 110 or attached with the piece of material 102. Furthermore, the top film 200 may be constructed of any suitable material for covering the opening 104, a non-limiting example of which includes a flexible clear plastic film.

Attached with a bottom portion 202 of the piece of material 102, is a bottom film 204. The bottom film 204 may be attached with the bottom portion 202 of the piece of material 102 through any suitable means for affixing two mediums together, non-liming examples of which include staples, tape, adhesive and being in-sewn. The bottom film 204 may be removed or otherwise manipulated to expose a bag 206, allowing the bag 206 to be extended from the piece of material 102. In addition to being attached with the piece of material 102, the bottom film 204 may be optionally attached with an exterior surface of the bag 206, such that pulling on the bottom film 204 pulls open the bag 206 and extends the bag 206 away from the opening 104. The bottom film 204 may be constructed of any suitable material for covering or protecting the bag 206, a non-limiting example of which includes a flexible clear plastic film.

As illustrated in FIG. 2B, the bag 206 is attached with the piece of material 102 and opening 104, such that an entrance to an interior 208 of the bag 206 is through the opening 104. The bag 206 may be attached with the piece of material 102 through any suitable means for affixing two mediums together, non-limiting examples of which include staples, tape, adhesive, in-sewn, and being compressed between multiple layers of the piece of material 102. The bag 206 may be constructed of any suitable material for constructing a bag, non-limiting examples of which include plastic, paper and cloth. The bag 206 has small perforations 210, allowing the bag 206 to breathe, such that when a user sneezes or coughs into the bag 206, air is allowed to pass through the small perforations 210. The small perforations 210 are medicated with an anti-septic, such that emissions are sterilized upon passing through the small perforations 210 from an interior 208 of the bag 206 to an exterior 212 of the bag 206. Additionally, the bag 206 may be any suitable size or shape. For example, the bag 206 has an entrance 214 and a base 216, where an area of the entrance 214 may be smaller than an area of the base 216. Furthermore, the bottom film 204 may optionally be attached with the bag 206, such that pulling on the bottom film 204 pulls open the bag 206.

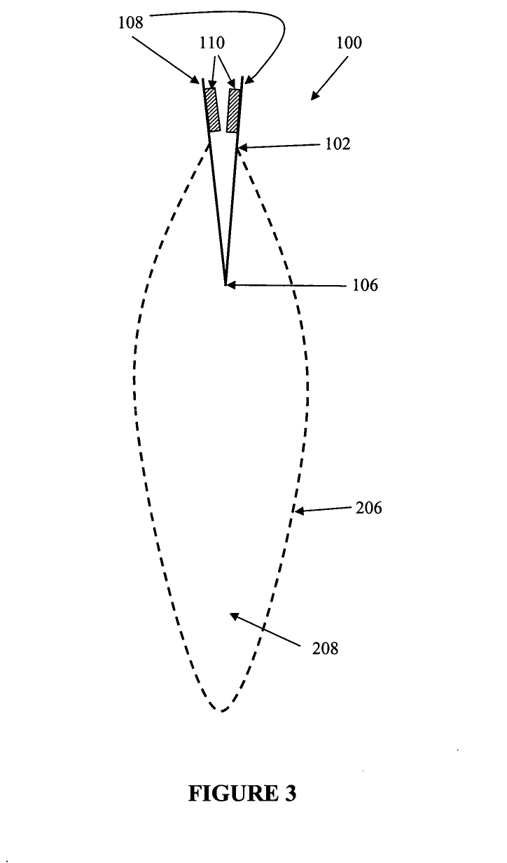

As illustrated in FIG. 3, the emission bag 100 may be sealed, containing any pathogens inside. When the piece of material 102 is folded along the perforations 106, the two opposing sides 108 are brought together. When the two opposing sides 108 are brought together, the enclosure apparatus 110 on the two opposing sides 108 come into contact with each other and thereby seal the emission bag 100. When sealed, any pathogens are trapped in the interior 208 of the bag 206.

1. An emission bag, for covering a nose and mouth in such a manner that pathogens transmitted from the nose and mouth will be confined to an interior of the emission bag, comprising:
a piece of material with an opening; and
a bag attached with the piece of material and opening, such that an entrance to an interior of the emission bag is through the opening.

2. An emission bag as set forth in claim 1, wherein a top film is attached with the emission bag, whereby manipulating the top film exposes the opening.

3. An emission bag as set forth in claim 1, wherein a bottom film is attached with a bottom portion of the piece of material, whereby manipulating the bottom film exposes the bag.

4. An emission bag as set forth in claim 1, wherein the piece of material is constructed from a material selected from a group consisting of plastic, metal, and paperboard.

5. An emission bag as set forth in claim 1, wherein the piece of material has perforations, allowing it to fold along the perforations and thereby creating two opposing sides.

6. An emission bag as set forth in claim 5, wherein the piece of material has an enclosure apparatus, such that when the piece of material is folded along the perforations, the enclosure apparatus on the two opposing sides come into contact with each other and thereby seal the emission bag.

7. An emission bag as set forth in claim 6, where the enclosure apparatus is selected from a group consisting of adhesive tabs, tape, Velcro, glue, and twist-ties.

8. An emission bag as set forth in claim 1, wherein the bag is constructed of a material selected from a group consisting of plastic, paper and cloth.

9. An emission bag as set forth in claim 1, wherein the bag has small perforations allowing the bag to breathe.

10. An emission bag as set forth in claim 9, wherein the small perforations are medicated with an anti-septic on the interior of the bag, such that emissions are sterilized upon passing through the small perforations from an interior of the bag to an exterior of the bag.

11. An emission bag as set forth in claim 1, wherein the bag has an entrance and a base and the entrance has an area and the base has an area, where the area of the entrance is smaller than the area of the base.

12. An emission bag as set forth in claim 2, wherein a bottom film is attached with a bottom portion of the piece of material, whereby manipulating the bottom film exposes the bag.

13. An emission bag as set forth in claim 12, wherein the piece of material is constructed from a material selected from a group consisting of plastic and paperpiece of material.

14. An emission bag as set forth in claim 13, wherein the piece of material has perforations, allowing it to fold along the perforations and thereby creating two opposing sides.

15. An emission bag as set forth in claim 14, wherein the piece of material has an enclosure apparatus, such that when the piece of material is folded along the perforations, the enclosure apparatus on the two opposing sides come into contact with each other and thereby seal the emission bag.

16. An emission bag as set forth in claim 15, where the enclosure apparatus is selected from a group consisting of adhesive tabs, tape, Velcro, glue, and twist-ties.

17. An emission bag as set forth in claim 16, wherein the bag is constructed of a material selected from a group consisting of plastic, paper and cloth.

18. An emission bag as set forth in claim 17, wherein the bag has small perforations allowing the bag to breathe.

19. An emission bag as set forth in claim 18, wherein the small perforations are medicated with an anti-septic, such that emissions are sterilized upon passing through the small perforations from an interior of the bag to an exterior of the bag.

20. An emission bag as set forth in claim 19, wherein the bag has an entrance and a base and the entrance has an area and the base has an area, where the area of the entrance is smaller than the area of the base.
